(19) United States
(12) Patent Application Publication
(10) Pub. No.: US 2006/0100576 A1
(43) Pub. Date: May 11, 2006

(54) Safety device of syringe needle

(76) Inventor: Tung-Hua Wang, Taipei County (TW)

Correspondence Address: Kang De Bao Biotechnology Co., Ltd., P.O. Box No. 6-57, Junghe, Taipei, 235, TW

(21) Appl. No.: 10/909,416

(22) Filed: Aug. 3, 2004

(51) Int. Cl. A61M 5/00; A61M 5/32

(52) U.S. Cl. 604/110; 604/192

(57) ABSTRACT

The present invention relates to a safety device of a syringe needle having a needle, a needle seat, and a needle lid. On the needle seat, a circular groove formed in the front and a breakable circular groove formed in the rear. At the end of the needle lid, a semicircle ring is attached. After injection, push the needle lid forward and insert the semicircular ring into the circular groove to secure the needle lid and then break the needle seat from the breakable circular groove, thereby preventing the needle from being re-used and enhancing the medical treatment safety.

### BACKGROUND OF THE INVENTION

(a) Field of the Invention

The present invention relates to a safety device of a syringe needle, and more particularly to a syringe in enhancing safety of the medical treatment pragmatically by preventing the syringe needle from being re-used and also avoiding a dangerous situation of being stabbed.

(b) Description of the Prior Art

Referring to FIG. 6. A conventional syringe includes a hypodermic needle 8, a needle seat 81, a syringe 9 and a lid 82, wherein, the hypodermic needle 8 is formed in the front of the needle seat 81 connecting to the syringe 9 and the lid 82 is formed to enclose the hypodermic needle 8 and the needle seat 81. To use the syringe, hold the lid 82 to put the needle seat 81 in a nozzle 91 of the syringe 9, and then detach the lid 82 to perform an injection, after drawing medicament. After the injection, the lid 82 is to be re-capped, a delicate action that a stab often happened to the nurse, causing a burden psychologically for being infected with bacteria after the stab. Furthermore, the re-capped needle may be detached from the lid, due to a non-secured structure, which may hurt people who dispose medical refuse. On the other hand, the used syringe may still be undamaged and often be re-used in developing and under-developed countries, which causes the spreading of contagious diseases.

### SUMMARY OF THE INVENTION

The present invention relates to a safety device of a syringe needle having a needle, a needle seat, and a needle lid. On the needle seat, a circular groove formed in the front and a breakable circular groove formed in the rear. At a rear end of the needle lid, a semicircle ring is attached. To use a syringe, move the needle lid backward to expose the needle. After injection, push the needle lid forward to enclose the needle and insert the semicircular ring into the circular groove to secure the needle lid and then snap the needle seat from the breakable circular groove, thereby preventing the needle from being re-used and enhancing the medical treatment safety.

To enable a further understanding of the said objectives and the technological methods of the invention herein, the brief description of the drawings below is followed by the detailed description of the preferred embodiments.

### BRIEF DESCRIPTION OF THE DRAWING

FIG. 1 shows an exploded elevational view of the present invention.

FIG. 2 shows the perspective view of the present invention.

FIG. 3 shows a cross-sectional view preparing for injection.

FIG. 4 shows a view inserting the semicircular ring into a circular groove.

FIG. 5 shows how to break a needle seat from a breakable circular groove.

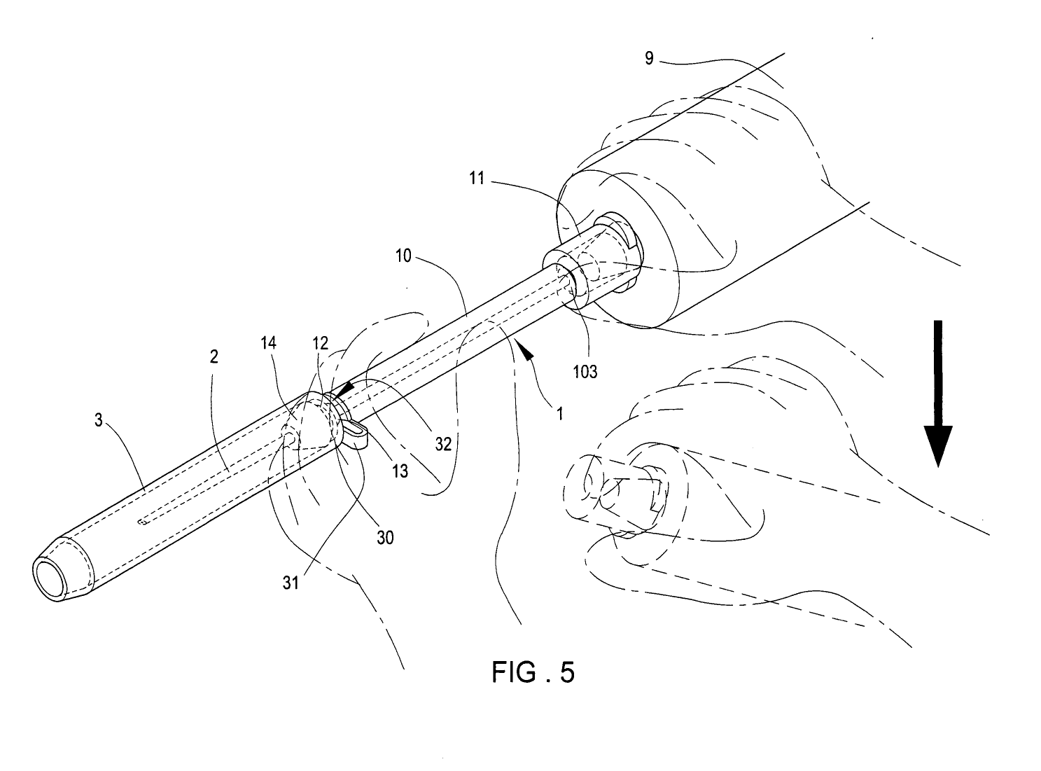

FIG. 6 shows a prospective view of a conventional syringe.

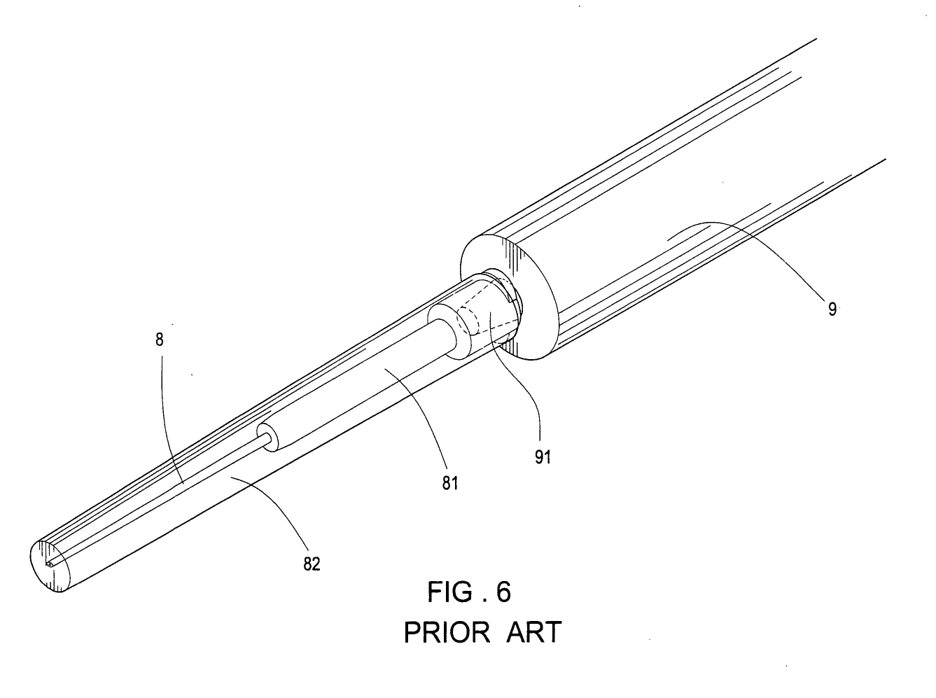

### DETAILED DESCRIPTION OF THE PREFERRED EMBODIMENTS

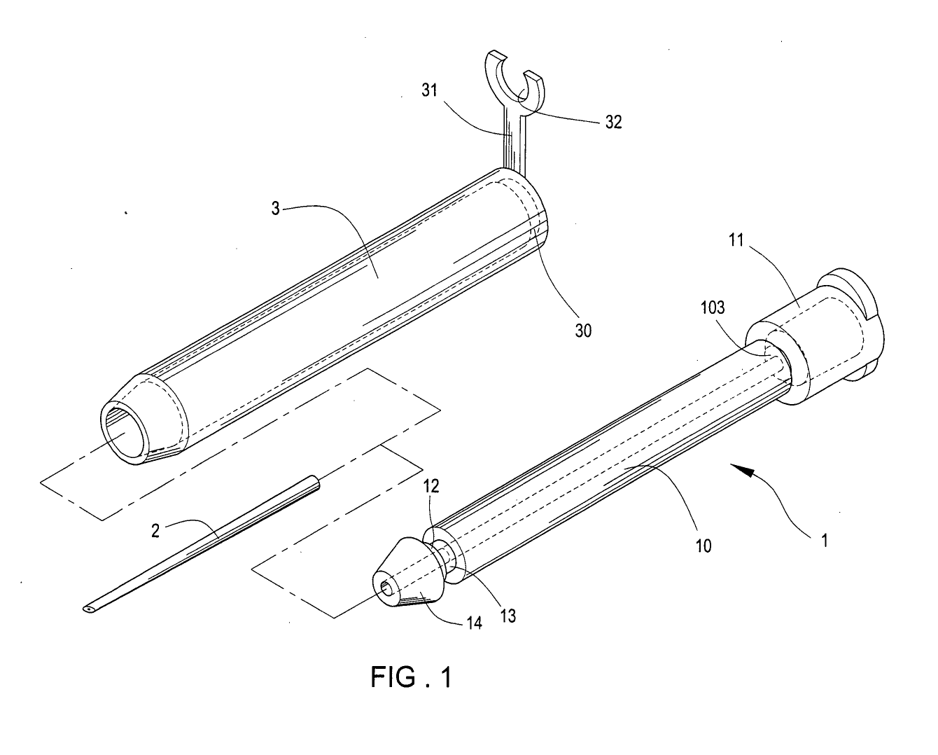

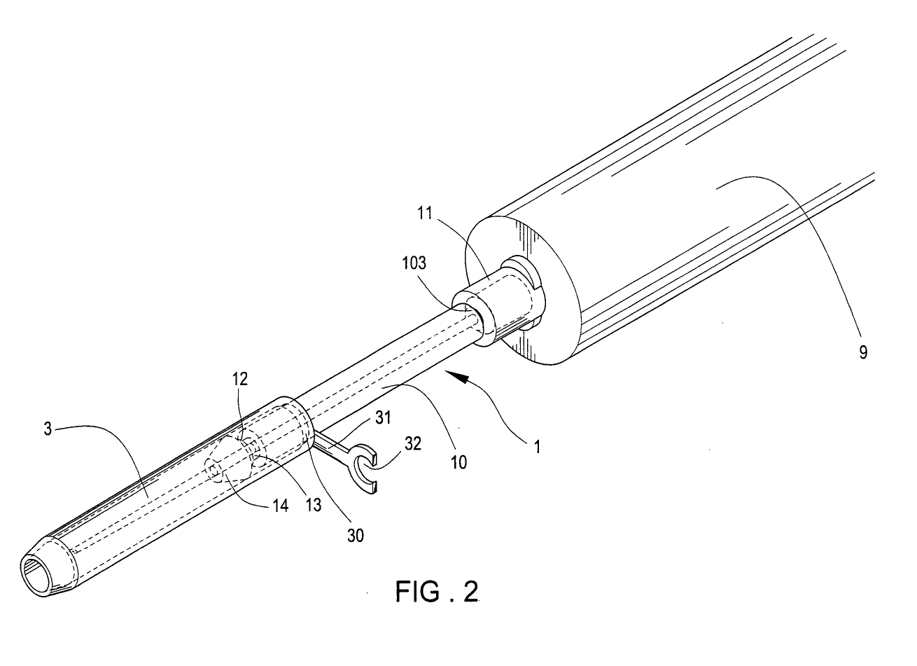

Referring to FIGS. 1 and 2. The present invention relates to a safety device of a syringe needle comprising a needle 2, a needle seat 1, and a needle lid 3.

The needle seat 1 is a cylinder 10 wherein a cone 14, a smaller ring 12 and a circular groove 13 formed in a row from the front end. The needle 2, projecting forwards, is inserted into a socket 11 formed in the rear of the cylinder 10, wherein, a connection is formed by a breakable circular groove 103.

The needle lid 3 for the cylinder 10 is also a cylinder, wherein, a projection ring 30, to be engaged with a the ring 12, formed on the inner perimeter and a semicircle ring 32 on a link 31 formed at the rear end, to be inserted into the circular groove 13.

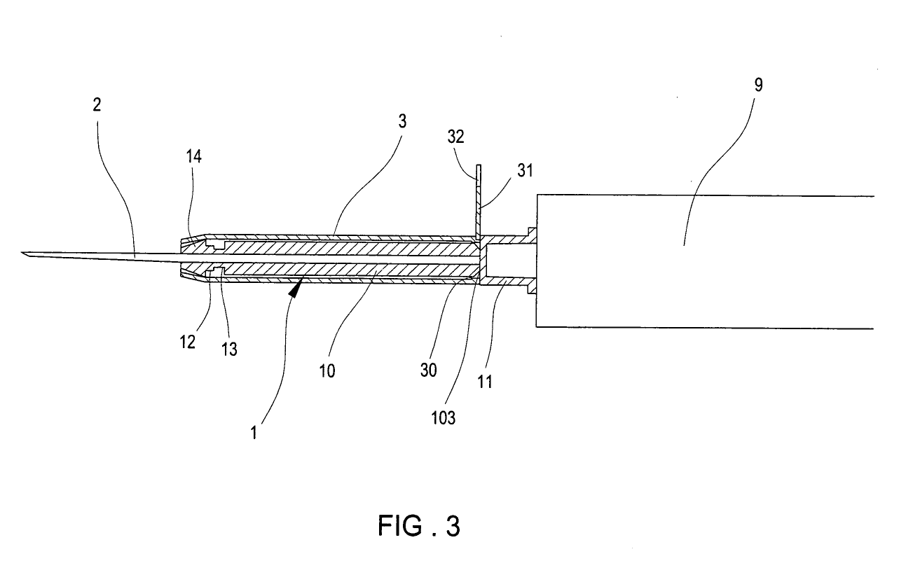

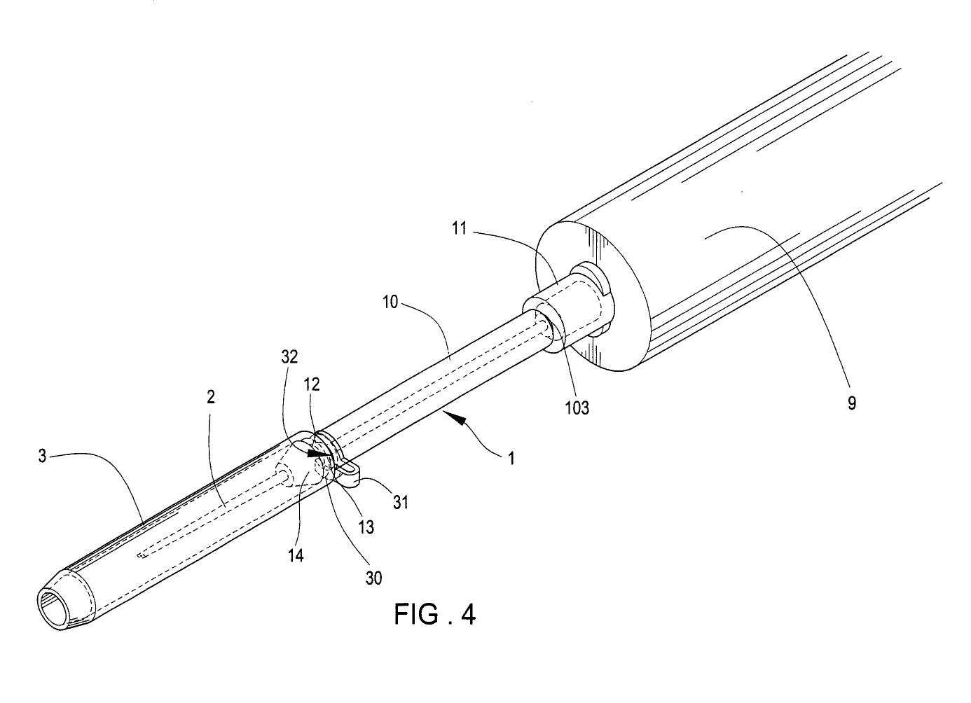

Referring to FIGS. 3 and 4. Before using, the needle lid 3 is positioned in the front of the needle seat 1 to enclose the needle 2, wherein the projection ring 30 of the needle lid 3, being on the ring 12, is blocked by a base of the cone 14, thereby preventing the needle lid 3 from moving away the needle seat 1. To use the syringe, move the needle lid 3 backward to expose the needle 2. After injection, push the needle lid 3 forward to the end to enclose the needle 2 and also expose the circular groove 13, then, by bending the link 31 backward down, to insert the semicircle ring 32 into the circular groove 13, thereby firmly secured the projection ring 30 of the needle lid 3 between a base of the cone 14 and the semicircle ring 32, preventing the needle lid 3 from moving backward. As such, risk of being stabbed when re-capping the needle lid from the front, as in the conventional syringe, is excluded.

Furthermore, the needle seat 1 can be broken, after using, by snapping the breakable circular groove 103, thereby preventing the needle 2 from being re-used.

In summary, the present invention is both advancement and practicality in enhancing safety of the medical treatment by preventing the needle from being re-used and also preventing a dangerous situation of being stabbed.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A safety device of a syringe needle comprising:
a needle seat, in the form of a cylinder, wherein, a cone, a smaller ring, and a circular groove formed in a row from the front end and a socket formed in the rear thereof;
a needle, projecting forwards, inserted into the needle seat thereof;
a needle lid for the needle seat, in the form of a cylinder; wherein a projection ring formed on an inner perimeter for engaging with the ring on the needle seat and a semicircle ring formed atop of a link connecting to an end, to be inserted into the circular groove formed behind the cone on the needle seat.

2. The safety device of a syringe needle as described in claim 1 wherein a breakable circular groove formed on the connection between the needle seat and the socket thereof.
